(19) United States
(12) Patent Application Publication
(10) Pub. No.: US 2021/0387016 A1
(43) Pub. Date: Dec. 16, 2021

(54) BLOOD IRRADIATION DEVICE AND METHODS FOR TREATING VIRAL INFECTIONS USING SAME

(71) Applicant: ASCEPI MEDICAL GROUP, LLC, Raleigh, NC (US)

(72) Inventor: John Scordos, Raleigh, NC (US)

(73) Assignee: ASCEPI MEDICAL GROUP, LLC, Raleigh, NC (US)

(21) Appl. No.: 17/343,596

(22) Filed: Jun. 9, 2021

Related U.S. Application Data
(60) Provisional application No. 63/037,253, filed on Jun. 10, 2020.

(51) Int. Cl. A61N 5/06

(52) U.S. Cl. CPC A61N 5/0624; A61N 2005/0661; A61N 2005/063

(57) ABSTRACT

An ultraviolet irradiation (UBI) device is configured to simultaneously irradiate blood flowing through a flat quartz crystal cuvette with both UV-A and UV-C light, wherein UV-A and UV-C lamps are positioned on opposite sides of the cuvette and calibrated to produce a two point source interference pattern having constructive interference points and maximum light intensity within the flow-through cuvette. Treatment of a cancer, an infectious disease or an autoimmune disease in a patient comprises simultaneously irradiating the blood with UV-A and UV-C light as it flows through the cuvette of the UBI device and into the vasculature of the patient.

### CROSS-REFERENCE TO RELATED APPLICATIONS

This application claims priority to and the benefit of U.S. Provisional Patent Application Ser. No. 63/037,253 filed Jun. 10, 2020 and entitled “Blood Irradiation Device and Methods for Treating Viral Infections Using Same,” the disclosure of which is incorporated herein by reference in its entirety for all purposes.

### FIELD

The present disclosure generally relates to medical devices and medical treatments, and in particular to an ultraviolet blood irradiation device and methods for treating various diseases and disorders using same.

### BACKGROUND

Photodynamic therapy (PDT) comprises targeted drug treatment where a photosensitive pharmaceutical active or other photosensitizer is used in conjunction with light in a therapy known as photodynamic therapy (PDT). In a formal sense, PDT requires both a photosensitizer and molecular oxygen. The photosensitizer is excited, resulting in the production of reactive oxygen species, such as singlet oxygen ¹O₂. PDT is unnecessarily intricate, and is narrowly applicable based on the nature of the photosensitizer chosen.

In view of the complexity of PDT, the field of medicine would benefit from medical devices capable of safely and efficiently irradiating blood samples with effective wavelengths in the ultraviolet spectrum without the need for a pharmaceutical active or other photosensitizer. Not only are novel medical treatments using only ultraviolet blood irradiation (UBI), (i.e., only light), still needed, the pharmacodynamics of UBI, particularly the oxidative mechanism of action of light on red and white blood cells in the absence of photosensitizing substances still needs to be understood and leveraged for new breakthrough therapies.

### SUMMARY

In accordance with various embodiments of the present disclosure, an ultraviolet blood irradiation (UBI) device for ultraviolet blood irradiation therapies is described. The UBI device irradiates blood flowing through a flat quartz crystal cuvette with UV-A and UV-C light simultaneously, wherein the UV-A and UV-C lamps providing the light are positioned on opposite sides of the flat quartz crystal cuvette, set to particular frequencies, and calibrated to produce a specific desired two point source interference pattern and associated electromagnetic field in and around the cuvette.

In various embodiments, the UBI device provides UV-A and UV-C irradiation of a blood sample. In various embodiments, a blood sample previously withdrawn from a patient is irradiated as it is infused back into the patient. In various embodiments, a blood sample from a blood bank is irradiated and then stored prior to use in a patient in need thereof

In various embodiments, the UBI device herein comprises a novel irradiation cuvette, comprising novel materials of construction that promote ultraviolet light transmission into the cuvette from both sides of the cuvette. In various embodiments, the UBI device of the present disclosure comprises a flat quartz crystal cuvette positioned such that UV-A light enters from one flat side and the UV-C light enters from the opposite flat side.

In various embodiments, the present disclosure provides a method of treating a disease or disorder in a subject in need thereof comprising the step of irradiating a previously withdrawn blood sample from the subject with UV-A and UV-C light as the blood sample flows through a flat quartz crystal flow-through cuvette.

In various embodiments, the present disclosure provides a method of treating a disease or disorder in a subject in need thereof comprising infusing compatible blood into the subject that was previously irradiated with UV-A and UV-C light as the blood sample flowed through a flat quartz crystal flow-through cuvette.

In various embodiments, the present disclosure also provides a method for treating a cancer, an infection, or an autoimmune disorder in a subject in need thereof. In this regard, both human and non-human animals having cancer, an infection, or an autoimmune disorder may be treated by ultraviolet blood irradiation of a blood sample from the subject in need thereof using the UBI device disclosed herein. In various embodiments, both human and non-human animals having a cancer, an infection, or an autoimmune disorder may be treated by infusing compatible blood that was previously subjected to ultraviolet blood irradiation using the UBI device disclosed herein.

In various embodiments, the present disclosure also provides a method for treating single-stranded RNA viral infections in a subject in need thereof. In this regard, both human and non-human animals having a viral infection caused by a single-stranded RNA virus may be treated by ultraviolet blood irradiation of a blood sample from the subject in need thereof using the UBI device disclosed herein.

In various embodiments, a method for treating a single-stranded RNA viral infection in a subject in need thereof comprises withdrawing a blood sample from the subject in need thereof, optionally adding saline, buffers and/or anticoagulants, and irradiating the blood sample with UV-A and UV-C light as the blood flows through a flat quartz crystal cuvette during re-infusion back into the subject. In various embodiments, the single-stranded viral infection in the subject in need thereof is a SARS-CoV-2 infection in a human subject, either asymptomatic or symptomatic.

In various embodiments, the UV-A light used in the UBI device has a wavelength of about 330-380 nm and the UV-C light used in the UBI device has a wavelength of about 245-265 nm. In various embodiments, the UV-C light used has a wavelength of about 254 nm.

### BRIEF DESCRIPTION OF THE DRAWING

The subject matter is pointed out with particularity and claimed distinctly in the concluding portion of the specification. A more complete understanding, however, may best be obtained by referring to the detailed description and claims when considered in connection with the following drawing figures:

FIG. 1 illustrates a UBI device in accordance with various embodiments of the present invention configured for simultaneous UV-A and UV-C irradiation of opposite sides of a flat cuvette;

FIGS. 2A-2C illustrate various embodiments of a flat quartz crystal cuvette for use in a UBI device according to the present disclosure;

FIG. 3 illustrates an arrangement of UV-A and UV-C lamps on opposite sides of a flat quartz crystal flow-through cuvette configured with tubing connections to each capillary end;

FIG. 4 illustrates a calibration of a two point source interference pattern resulting from UV-A and UV-C electromagnetic radiation sources aimed at each other and aimed at opposite sides of a flat cuvette;

FIG. 5 sets forth a method of treating a disease state in a subject in need thereof using a UBI device in accordance with the present disclosure; and

FIG. 6 illustrates a method of treating a disease, infection, or disorder by simultaneously irradiating a previously withdrawn blood with both UV-A and UV-C radiation as the blood is infused back into the patient through the UBI device and into a placed catheter.

### DETAILED DESCRIPTION

The detailed description of exemplary embodiments makes reference to the accompanying drawings, which show exemplary embodiments by way of illustration and their best mode. While these exemplary embodiments are described in sufficient detail to enable those skilled in the art to practice the invention, it should be understood that other embodiments may be realized and that logical, chemical, and mechanical changes may be made without departing from the spirit and scope of the inventions. Thus, the detailed description is presented for purposes of illustration only and not of limitation. For example, unless otherwise noted, the steps recited in any of the method or process descriptions may be executed in any order and are not necessarily limited to the order presented. Furthermore, any reference to singular includes plural embodiments, and any reference to more than one component or step may include a singular embodiment or step. Also, any reference to attached, fixed, connected or the like may include permanent, removable, temporary, partial, full and/or any other possible attachment option. Additionally, any reference to without contact (or similar phrases) may also include reduced contact or minimal contact.

In various embodiments of the present disclosure, a UBI device is disclosed. The UBI device comprises a cuvette configured to contain a static or flowing blood sample, a UV-A light source and a UV-C light source. In various embodiments, the cuvette comprises a flat quartz crystal flow cell.

### Definitions and Interpretations:

As used herein, the terms “ultraviolet light” and “ultraviolet radiation” interchangeably refer to that portion of the electromagnetic spectrum from about 100 to about 400 nm in wavelength. The common terms of “vacuum UV” (100-200 nm), “UV-C” (200-280 nm), “UV-B” (180-315 nm) and “UV-A” (315-400 nm) light may also be referred to throughout the present disclosure.

As used herein, the term “cuvette” refers to an enclosure configured for UV irradiation of its contents, comprising at least one wall transparent to at least a portion of the UV spectrum. In various embodiments, a cuvette comprises walls that define one or more exterior surfaces, and that together define an interior cavity configured to hold a volume of blood. In various embodiments, a cuvette herein may further comprise any number of ports, such as one or more inlets and one or more outlets, and/or one or more ports configured as valves or septa for introducing liquids, gasses, or solids into the interior cavity of the cuvette, and which can participate in a flow-through configuration. For example, a cuvette herein may comprise any combination of quick-disconnect fitting such as Luer, ball valves, and resilient septa, or may simply comprise tapered capillary ends that can be friction fitted with rubber tubing. In various embodiments, a flow-through cuvette comprises an inlet, an outlet, and an interior, all in fluidic communication, such that a liquid, such as blood, can be moved into the inlet, through the interior of the cuvette, and out the outlet in a continuous flow. A cuvette herein may be an assemblage of multiple components, such as square panels and gaskets that may be disassembled for cleaning, or may comprise a single contiguous structure, such as a cuvette consisting of a glass tube. In various embodiments, a cuvette for use herein consists entirely of quartz crystal, and may be in the shape of a flattened tube with ends that are drawn into tubing connectors. In various embodiments herein, a cuvette may be similar to a quartz flow cuvette cell used for various flow-through measurements, having a flat shaped interior, one inlet and one outlet. In various embodiments, a flat glass cuvette may be constructed from “UV glass,” also referred to as Suprasil® (various grades, available from Heraeus Group, Hanau, Germany), which is a synthetic fused silica glass (≥99.9% SiO₂) exhibiting optical transmission of >60% through 1 mm thickness from about 190 to about 2500 nm. An alternative is “Normal Clear Tube” (PQ181) from Jiangsu Pacific Quartz Co., Ltd., Jiangsu, China. Custom glass products made from PQ181 tubes are available from John Moncrieff, Ltd., Kinross, Scotland.

As used herein, the term “UV lamp” is used broadly to include any UV light source, regardless of whether the source resembles a traditional incandescent bulb or not. Non-limiting examples of UV lamps include mercury vapor lamps, halogen lights, light-emitting diodes (LEDs), gas discharge lamps, electrical arcs, black lights, fluorescent lamps, incandescent lamps, UV lasers, and plasma and synchrotron UV sources. For simplicity, a UV light source used herein may be referred to simply as a “lamp.” UV lamps are available, for example, from Aquafine Corporation, Valencia, Calif., USA.

As used herein, the terms “subject,” “subject in need thereof,” “patient,” or “patient in need thereof,” each refer to any human or non-human animal (i.e., a mammal) from which a blood sample may be withdrawn and irradiated with UV light, or that may be treated with previously irradiated blood provided, for example, from a blood bank. Non-limiting examples of a subject herein include a human of any age or gender, a dog, a cat, a rabbit, a cow, a porcine and a bird. In general, a subject herein in need of UBI therapy includes humans along with both domesticated pets, zoo animals, and farm animals.

As used herein, the term “blood sample” is used broadly to include both whole blood as occurring in the subject, e.g., freshly withdrawn from the human or non-human subject, but also any blood sample that has been diluted or otherwise changed by adding various substances. In various embodiments, a blood sample herein, such as a blood sample readied for UBI, may be treated with saline, salts, buffers, anticoagulants, or other pharmaceutical agents. Thus, for simplicity, both a blood sample withdrawn from a subject and that blood sample subsequently modified by saline and coagulant may be referred to as the “blood sample.” Further, a blood sample need not be freshly withdrawn from a person or non-human animal in need of UBI treatment. Blood samples may be part of a standard blood bank, and from time to time, blood samples may be irradiated in accordance with the present disclosure and then returned to storage in the blood bank until the need arises for previously irradiated blood. Herein, reference may be made to “compatible blood,” which simply means blood acceptable for infusion into a person or non-human animal in need thereof, such as, for example, blood having the same blood type and/or species origin as the person or non-human animal.

As used herein, the term “treatment” or “treating” generally refers to an intervention in an attempt to alter the natural course of the human or non-human subject being treated, and can be performed either for prophylaxis or during the course of clinical pathology. Desirable effects include, but are not limited to, preventing occurrence or recurrence of a disease or disorder, alleviating symptoms, suppressing, diminishing, or inhibiting any direct or indirect pathological consequences of the disease or disorder, ameliorating, or palliating the disease state or the disorder, and causing remission or improved prognosis. Treatment herein also includes modulating an immune response against one or more antigens, such as pathogens or genetic material therefrom. In various embodiments, modulating the immune response comprises eliciting, stimulating, inducing, promoting, increasing, or enhancing the immune response against one or more antigens. In various aspects, the UBI device and therapies disclosed herein elicits, stimulates, induces, promotes, increases, or enhances an immune response against a species or subspecies of Coronavirus. In various aspects, the UBI device and therapies disclosed herein elicits, stimulates, induces, promotes, increases, or enhances an immune response against a species or subspecies of Ebola virus. In embodiments, the UBI device and therapies disclosed herein elicits, stimulates, induces, promotes, increases, or enhances an immune response against both a species or subspecies of Coronavirus and a species or subspecies of Ebola virus. In various embodiments, the UBI device and therapies herein elicits, stimulates, induces, promotes, increases, or enhances an immune response against a species or subspecies of Coronavirus. In various embodiments, the UBI device and therapies disclosed herein elicits, stimulates, induces, promotes, increases, or enhances an immune response against a species or subspecies of Ebola virus. In various embodiments, the UBI device and therapies disclosed herein elicits, stimulates, induces, promotes, increases, or enhances an immune response against both a species or subspecies of Coronavirus and a species or subspecies of Ebola virus. In various embodiments, the various diseases and disorders treated by UBI therapy include, but are not limited to, a cancer, an infectious disease, and an autoimmune disease or disorder.

As used herein, the term “cancer” takes on its ordinary meaning in medicine and is not meant to be limiting in regard to type. A human or non-human animal may have, for example, bladder cancer, breast cancer, colorectal cancer, kidney cancer, lung cancer, lymphoma, melanoma, various oral cancers, and so forth, which can be treated by infusing blood into the human or non-human animal in need thereof that was irradiated by UV light in accordance to the present disclosure. A comprehensive listing of cancers may be found at https://www.cancer.gov/types, and any one or combination of these disease states in humans or non-human animals may be treated by UBI treatment using the blood irradiation device herein.

As used herein, the term “infectious disease” refers to any disease that is caused by an infectious agent. An “infectious agent” includes any exogenous pathogen including, without limitation, bacteria, fungi, viruses, mycoplasma, and parasites. Infectious agents that may be treated with UBI therapy per the present disclosure include any art-recognized infectious organisms that cause pathogenesis in an animal, including such organisms as bacteria that are gram-negative or gram-positive cocci or bacilli, DNA and RNA viruses, including, but not limited to, DNA viruses such as papilloma viruses, parvoviruses, adenoviruses, herpesviruses and vaccinia viruses, and RNA viruses, including single-stranded RNA viruses, such as, for example, arenaviruses, coronaviruses, rhinoviruses, respiratory syncytial viruses, influenza viruses, picomaviruses, paramyxoviruses, reoviruses, retroviruses, and rhabdoviruses. Examples of fungi that may be treated with UBI therapy in accordance with the present disclosure include fungi that grow as molds or are yeastlike, including, for example, fungi that cause diseases such as ringworm, histoplasmosis, blastomycosis, aspergillosis, cryptococcosis, sporotrichosis, coccidioidomycosis, paracoccidio-idomycosis, and candidiasis. UBI therapy provided for herein may also be utilized to treat parasitic infections including, but not limited to, infections caused by somatic tapeworms, blood flukes, tissue roundworms, ameba, and Plasmodium, Trypanosoma, Leishmania, and Toxoplasma species.

As used herein, the term “single-stranded RNA virus” takes on its ordinary meaning in virology to include both positive-sense RNA viruses (Group IV) and negative-sense RNA viruses (Group V). The Group IV viruses include such well-known pathogens as hepatitis C, West Nile, dengue, MERS, and SARS, including SARS-CoV-2. The Group V viruses also include well-known pathogens, the most infamous perhaps being Ebola. For simplicity, the terms “Groups IVN viruses” or “ssRNA viruses” may be used interchangeably with one another and interchangeably with the term fully written out terms “positive-sense single-stranded RNA viruses” and “negative-sense single-stranded viruses.” The UBI therapies in accordance with the present disclosure are particularly effective at treating an infectious disease in a human or non-human animal caused by a single-stranded RNA virus.

In various embodiments of the present disclosure, the infectious disease to be treated by UBI therapy in conjunction with the blood irradiation device disclosed herein is caused by a Coronavirus. In various embodiments, the Coronavirus at issue is selected from a species or subspecies of Embecovirus, Sarbecovirus, Merbecovirus, Nobevovirus, Hibecovirus, SARSr-CoV, or MERS-CoV. In various embodiments, the Coronavirus is selected from a species or subspecies of SARS-CoV, SARS-CoV-2, MERS-CoV, SL-CoV-WIV1, HK84, HKUS, HCoV-0C43, HCoV-HKU1, or HKU9. In embodiments, the

Coronavirus comprises an alphacoronavirus, a betacoronavirus, a gammacoronavirus, or a deltacoronavirus protein or peptide or a variant, homologue, derivative or subsequence thereof The UBI therapies in accordance with the present disclosure are particularly effective at treating an infectious disease in a human or non-human animal caused by a Coronavirus.

As used herein, “autoimmune disease or disorder” takes on its ordinary meaning in medicine, and is not meant to be limiting in any way with regard to type of disease or disorder. Various autoimmune diseases that may be treated by UBI therapy in conjunction with the blood irradiation device of the present disclosure are found, for example, at https://www.aarda.org/diseaselist/, where there are over 100 autoimmune diseases listed and relevant to the present disclosure. For example, UBI therapies in accordance with the present disclosure are particularly effective at treating autoimmune diseases such as multiple sclerosis, myasthenia gravis, pernicious anemia, reactive arthritis, rheumatoid arthritis, lupus, chronic Lyme disease, type-1 diabetes, endometriosis, fibromyalgia, and sarcoidosis, amongst many others and combinations of these and other autoimmune diseases.

### General Embodiments of the UBI Device and Methods of Using Same

In various embodiments, a UBI device in accordance with the present disclosure comprises: a cuvette; a UV-A lamp configured to irradiate material present within the cuvette with UV-A light; and a UV-C lamp configured to irradiate material present within the cuvette with UV-C light. In various embodiments, the UBI device is configured for sequential UV-A and UV-C blood irradiation, in either order, or configured for the simultaneous irradiation of blood with both UV-A and UV-C light.

In various embodiments, the cuvette comprises a flat quartz crystal flow cell dimensionally configured for the simultaneous irradiation of material contained inside or flowing therethrough with the UV-A light and the UV-C light. In various embodiments, a first flat side of the flat quartz crystal cuvette is irradiated with UV-A light while a flat, opposite second side of the flat quartz crystal cuvette is simultaneously irradiated with UV-C light.

In various embodiments, the UV-A lamp and the UV-C lamp are disposed on opposites sides of a flat crystal cuvette such that the UV-A incident radiation and the UV-C incident radiation are aligned and generally aimed toward each other to generate a particular magnetic field in and around the cuvette due to the electromagnetic wave interference patterns. The distance from each UV lamp to the cuvette is adjusted to achieve the desired interference pattern and associated electromagnetic field in and around the cuvette. In various embodiments, this calibration comprises adjustment of the ballasts for the lamps, i.e., electronic calibration of the lamps themselves rather than mechanical calibration through distances between the lamps and from each lamp to the cuvette.

General aspects of a UBI device in accordance with the present disclosure and its components will become more evident in the discussions of the various drawing figures.

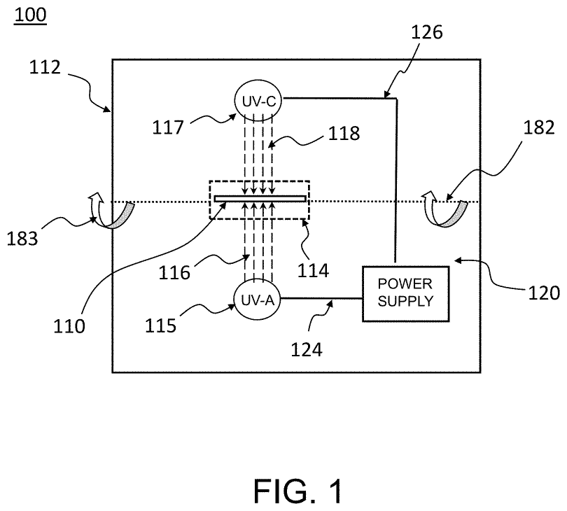

With reference now to FIG. 1, a UBI device 100 according to the present disclosure comprises a cuvette 110, a UV-A lamp 115 configured to irradiate material present within the cuvette 110 with UV-A light 116, and a UV-C lamp 117 configured to irradiate the material present within the cuvette 110 with UV-C light 118. In various embodiments, the cuvette comprises a flat quartz crystal cuvette described in more detail herein below. In FIG. 1 the physical details of the cuvette 110 are not illustrated for purposes of clarity. In various embodiments, and as shown in FIG. 1, the UBI device 100 is configured for the simultaneous irradiation of the cuvette 110 with both UV-A and UV-C light impinging on and through the cuvette from opposite sides.

The UBI device 100 may further comprise an exterior housing 112, such a metal box, to contain and organize these and other components. The particular cross-sectional shape of the exterior housing 112 shown for the UBI device 100 (i.e., square in FIG. 1) should not be interpreted as limiting. Further, the UBI device 100 may comprise two separate, yet connected, housings 112 that might move relative to one another through a hinge, such as to allow insertion of the cuvette 110. For example, the UV-A lamp 115 and the UV-C lamp 117 may be housed in separate portions of the overall device. In various embodiments, a UBI device comprises two metal boxes, one containing the UV-A lamp and the other containing the UV-C lamp, and the two metal boxes are hinged together (represented by the dashed line 182) such that the box on the bottom can remain stationary while the one on the top can hinge back (by motion 183) to reveal a receptacle 114 for receiving a cuvette (discussed further herein below). Although the lamp arrangement between top and bottom boxes is somewhat arbitrary, an advantage to configuring the UV-A light in the bottom stationary box is that when opening the device by moving the top box through the hinged connection 182, one doesn't accidentally stare down into UV-C light shining up, which is very dangerous to eyesight. So, in the ideal configuration, a UBI device comprises a bottom housing containing the UV-A lamp and a top housing containing the UV-C lamp, with the two housings hingably connected to each other. A user simply opens the UBI device 100 like a sandwich press to access the cuvette receptacle 114, places the flat cuvette in the receptacle 114 between the housings, and then closes the top housing back onto the bottom housing to begin irradiation. This hinging arrangement also provides easy access to the cuvette so it can be periodically flipped over.

A housing 112 for these and other electrical components may comprise metal and/or plastic walls, e.g., in the form of panels, configured into an enclosure having an interior space and exterior surfaces defined by the various panels. The area outside of the housing 112 may be referred to as the environment exterior to the UBI device 100, or simply the exterior environment. As explained in more detail herein, the housing 112 may comprise any number of hinging, sliding, folding, or roll-up doors usable to access interior portions of the UBI device 100. Attached to any panel of the housing 112, or in close proximity to a vent configured therethrough, may be one or more cooling fans for moving heated air from within the interior of the UBI device 100 to the exterior environment. These and other cooling fans may be associated with, and in close proximity to, the UV-A lamp 115 and/or the UV-C lamp 117, such as to move the heat generated from these lamps to the exterior environment so as to mitigate unwanted heating of the cuvette 110. Not illustrated in FIG. 1 are other components that may be included on the exterior surfaces of the UBI device 100, such as for example, an electrical cord for providing power to the UBI device 100, various knobs and switches, indicator lights, decals, and so forth.

With continued reference to FIG. 1, the UBI device 100 further comprises a receptacle 114 configured for reversibly containing the cuvette, into which the cuvette can be placed, so as to ensure alignment of the cuvette 110 with both the UV-A light 116 and the UV-C light 118 emanating from opposite sides of the cuvette 110. In various embodiments, the cuvette 110 may be flipped, such as by rotation along its axis, to reverse which side of the cuvette 110 receives which incident light, i.e., the UV-A 116 or the UV-C 118 light. In various embodiments, the cuvette 110 may include a cuvette holder, such as a frame, into which the cuvette 110 fits in order to ensure correct alignment of the cuvette. Not illustrated in FIG. 1 are fluidic connections to the cuvette 110 configured for passing a liquid sample through the cuvette during irradiation.

With continued reference to FIG. 1, the UBI device 100 may further comprise a power supply 120 and the necessary electrical connections 124 from the power supply 120 to the UV-A lamp 115, and electrical connections 126 from the power supply 120 to the UV-C lamp 117. The power supply may be configured to provide whatever voltage, phase and amperage needed to power the lamps, and to power any other electronic components within the UBI device 100 requiring electrical power. For example, the power supply 120 may provide AC or DC voltage to the lamps 115 and 117 depending on the requirements of the UV lamps. Any additional componentry, such as transformers, rectifiers, ballasts, relays, switches, indicator lights, fans, bus bars, power supply cords, timers, keyed lock-out safety switches, and their associated wiring and circuit boards, are not illustrated in FIG. 1 for purposes of clarity. In various embodiments, the UBI device 100 may comprise any combination of these components.

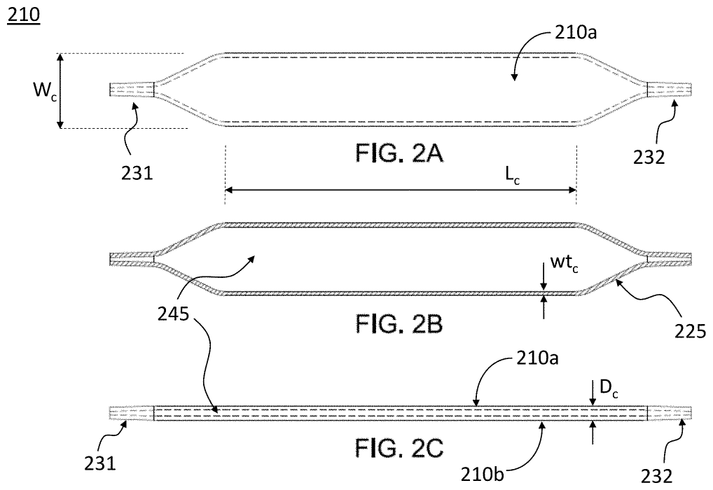

With reference now to FIGS. 2A-2C, a UBI device in accordance with the present disclosure comprises a cuvette that has at least some transparency to UV light. The cuvette 210 illustrated in FIGS. 2A-2C, which is the preferred cuvette for the UBI device and methods using same as disclosed herein, is made entirely of quartz crystal, and is therefore substantially transparent to UV light. This material was described in the definitions section above. FIG. 2A illustrates a plan view of the cuvette, where a first flat side is visible. The second, opposite flat side would be underneath the drawing, and would be identical to the first flat side. The cuvette 210 further comprises drawn capillary ends 231 and 232 as illustrated. The dashed lines in FIG. 2A represent the inner wall surfaces. In various embodiments, the flat quartz cuvette 210 is configured to have a width W_{c}, a length between curved ends L_{c}, a depth D_{c}, and a glass wall thickness of wt_{c}. The pathlength of a cuvette is the distance between the interior walls in the direction of the light path through the cuvette, so as illustrated, the cuvette pathlength is D_{c}−(2×wt_{c}). Ranges for these dimensions are discussed below, and it should be understood that dimensions outside the ranges indicated herein may become necessary as cuvettes are optimized in UBI treatments of various disease states not previously contemplated.

To make such a flow-through cuvette with a flat shape, a quartz crystal tube, such as a piece of PQ181 having a wall thickness wt_{c} of from about 1.0 mm to about 2.0 mm, and preferably about 1.5-1.75 mm, is heated to soften the glass and the softened glass is then compressed to a point where the D_{c} depth dimension is less than about 10 mm, and the volume remaining inside the flattened tube is substantially reduced, e.g., to less than about 10 cc, and preferably less than about 5 cc. In a separate step, the glass can be heated at each end, and each of the softened ends drawn into a capillary finish configured to accept friction fitting of flexible tubing such as catheter lines. The capillary ends 231 and 232 may have an inner diameter of about 1-3 mm, preferably about 2 mm, and an outer diameter of about 3-8 mm, preferably about 5 mm. This example ordering of steps is not intended to be limiting, as a glass manufacturer may have their own process for converting a piece of PQ181 tube into the flat flow-through quartz crystal cuvette 210 illustrated in FIGS. 2A-2C, such as drawing the capillary ends first, and then flattening the tube into a flat shape. Further, there may be any number of subsequent annealing steps to reduce or eliminate any mechanical stress related birefringence in the optical glass.

In various embodiments, the overall dimensions of the flat cuvette 210 are such that the cuvette has an interior capacity of about 0.1-10 cc, preferably about 1-5 cc, with a wall thickness wt_{c} of about 1.5-1.75 mm. In various embodiments, D_{c} is about 3-6 mm and preferably about 4.1 mm to about 4.6 mm. Therefore, with a wall thickness wt_{c} of from about 1.5 mm to about 1.7 5mm, the pathlength for a cuvette of use herein is from about 0.6 mm to about 1.6 mm. In various embodiments, and as illustrated in FIGS. 2A-2B, the sides are rounded and the cuvette finished at a width W_{c} of about 20-30 mm, preferably about 25.4 mm (1 inch) and at a length Lc between the curved ends of about 100-150 mm, preferably about 127 mm (5 inches). As mentioned, a flat cuvette for use herein may be configured with any of the various dimensions outside these ranges. For example, the dimensions of a custom cuvette may be configured in response to the shape and dimensions of particular UV lamps, or in response to the nature of the disease state requiring UBI treatment, or in response to targeting a particular flow rate through the cuvette during irradiation, and/or in response to targeting a particular irradiation dwell time in the cuvette. Furthermore, patient variability may necessitate custom cuvette dimensions for a particular patient, even when patients have the same disease or infection.

FIG. 2B is a cross-sectional view of the cuvette 210, illustrating the interior cavity 245 and glass walls 225 having a wall thickness wt_{c} of from about 1.0 mm to about 2.0 mm, and preferably about 1.5-1.75 mm.

FIC. 2C is an end view of the cuvette 210 to illustrate the overall flat shape, the interior cavity 245, and the capillary ends 231 and 232 for connecting flexible tubing to the cuvette 210. The cuvette 210 comprises a first flat side 210a and second flat side 210b opposite the first flat side 210a. The depth Dc of the cuvette is about 3-6 mm and preferably about 4.1 mm to about 4.6 mm.

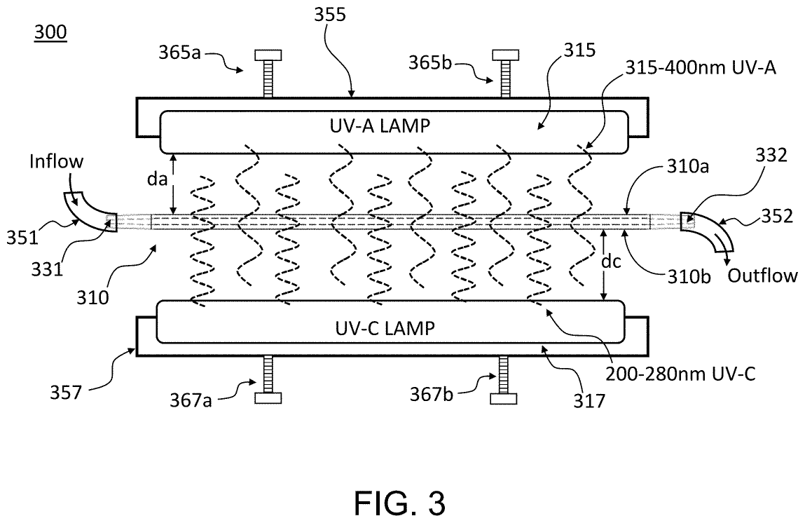

FIG. 3 illustrates the relative positioning of the flat quartz crystal flow-through cuvette 310 between the two UV lamps, in accordance with various embodiments of the present disclosure. FIG. 3 is meant to be generic in the sense that detailed componentry such as the frames for holding each of the lights, adjustment screws to move each light frame closer or further from the cuvette 310 to calibrate the UBI device, and the distances da and dc shown, are all variable. All other componentry within the UBI device is omitted for purposes of clarity.

With reference to FIG. 3, a portion of the UBI device 300 comprises the flat quartz crystal cuvette 310, the UV-A lamp 315, and the UV-C lamp 317. As mentioned, the essence of the UBI device is the simultaneous radiation of UV-A and UV-C light into the flat cuvette 310 from opposite sides of the cuvette.

The UV-A lamp 315 may be housed in a frame 355, with the frame optionally providing electrical connections to power the UV-A lamp 315 and one or more fans to remove heat from the lamp. As illustrated, the UV-A lamp frame 355 may further comprise one or more adjustment screws 365a, 365b, and so forth, configured to move the lamp frame 355 closer to, or further away from, the cuvette 310. In the configuration shown, turning the adjustment screws 365a and 365b move the UV-A lamp frame 355, changing the distance da from the UV-A lamp to the first flat face 310a of the cuvette 310. Other structures may be included such that the screws can turn in one structure and push or pull the UV-A light frame 355.

Similarly, the UV-C lamp 317 may be housed in a lamp frame 357, with the frame optionally providing electrical connections to power the UV-C lamp 317 and one or more fans to remove heat from the lamp. As illustrated, the UV-C lamp frame 357 may further comprise one or more adjustment screws 367a, 367b, and so forth, configured to move the lamp frame 357 closer to, or further away from, the cuvette 310. In the configuration shown, turning the adjustment screws 367a and 367b move the UV-C lamp frame 357, changing the distance dc from the UV-C lamp to the second, opposite flat face 310b of the cuvette 310. Other structures may be included such that the screws can turn in one structure and push or pull the UV-C light frame 357.

In various embodiments, calibration of the blood irradiation device comprises optimizing the distances da and dc to maximize light intensity at the position that is or will be occupied by the internal flow chamber of the cuvette. In various embodiments, the distances da and dc are substantially the same. In various embodiments, the distances da and dc are different. In various embodiments, the distance da from the UV-A lamp to a flat side of the cuvette is from about 0.75 inches (about 19 mm) to about 1.25 inches (about 31.75 mm). In various embodiments, the distance dc from the UV-C lamp to the opposite flat side of the cuvette is from about 0.75 inches (about 19 mm) to about 1.25 inches (about 31.75 mm).

As shown in FIG. 3, the flat quartz crystal cuvette 310 is configured with capillary ends 331 and 332, as per FIGS. 2A-2C. In various embodiments, inlet tubing 351 is connected to capillary end 331, and outlet tubing 352 is connected to capillary end 332, by friction fit between complimentary diameters. These connections can be reversed since the cuvette 310 is rotationally symmetrical. However, it may be desired to arrange inflow and outflow as shown to take advantage of gravity rather than a mechanical pump to move a liquid sample through the cuvette 310 for irradiation. In various embodiments, a bag containing a blood sample is positioned above the inflow to capillary end 331, and a patient's vasculature is positioned below the outflow from capillary end 332, such that the blood sample contained in the bag can be infused back into the patient as it flows through the cuvette 310 to be simultaneously irradiated with UV-A and UV-C light. It is important to understand that in various embodiments, the human or non-human patient need not be present at all during the irradiation procedure, only a blood sample need be present. For example, a bag containing a blood sample may be retrieved from storage, such as from a blood bank, connected to and positioned above the inflow to capillary end 331, and another receptacle, such as another bag or a vacuum bottle, connected to and positioned below the outflow from capillary end 332, such that the blood sample contained in the first bag can be irradiated and captured in the second bag or bottle where it can be stored away again in the blood bank for future use. In this way, therapeutic blood samples can be available for use in blood infusions when needed.

With continued reference to FIG. 3, the UV-A lamp 315 emits UV-A radiation having wavelength between 315-400 nm towards and through the cuvette 310 and its contents. The UV-C lamp 317 emits UV-C radiation having wavelength between 200-280 nm towards and through the cuvette 310 and its contents. In various embodiments, the UV-C lamp emits UV-C radiation having wavelength between 245-265 nm. In various embodiments, the UV-C lamp comprises a mercury vapor lamp. In various embodiments, the mercury vapor UV-C lamp may comprise a medium pressure (MP) polychromatic lamp. In various embodiments, the mercury vapor UV-C lamp may comprise a low pressure (LP) monochromatic lamp emitting essentially a single wavelength of UV-C light, namely 254 nm.

As illustrated in FIG. 3, the UV-A lamp and the UV-C lamp are configured on opposite sides of the flat quartz crystal cuvette 310. In this configuration, the UV-A longer wavelength electromagnetic radiation and the UV-C shorter wavelength electromagnetic radiation are directionally aimed at each other and through the cuvette 310. Stated another way, the UBI device 300, in accordance with various embodiments of the present disclosure, comprises two electromagnetic radiation sources positioned to create an interference pattern symmetrically disposed in and around the axis of the cuvette 310.

### Blood Irradiation Device Calibration

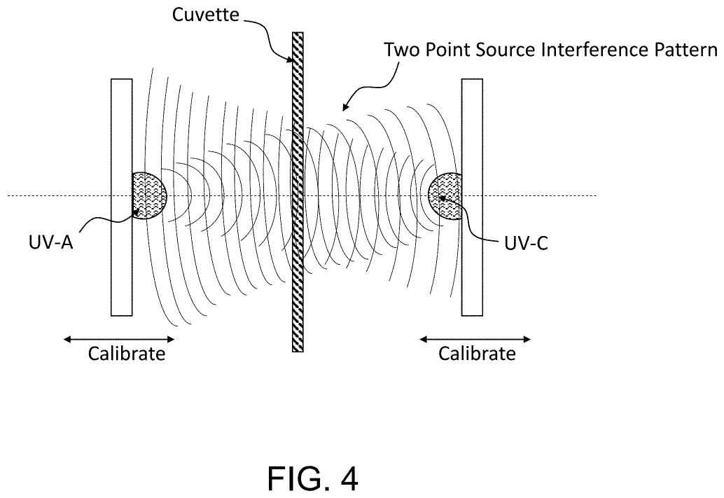

As illustrated in FIG. 4, the UV-A and UV-C lamps are calibrated to induce a specific and desired electromagnetic “two point source” interference pattern, recognizing that the wavelengths emanating from each lamp are necessarily different. The pattern can be optimized such that constructive interference points rather than cancelling nodes or destructive interference are found centered in the cuvette. The term “centered” means that the constructive interference points are disposed within the internal cavity of the flow-through cuvette such that blood flowing therethrough will necessarily be subjected to the additive waves. In various embodiments, calibration comprises moving either or both lamps along the axis indicated by the dashed line, further from, or closer to, the cuvette. As mentioned, mechanical calibration comprises optimizing the distances da and dc for constructive interference in the flow path of the cuvette by moving the lamps relative to the cuvette.

In various embodiments, the UV-A lamp is moved such that the calibrated distance da is from about 0.75 inches (about 19 mm) to about 1.25 inches (about 31.75 mm), and the UV-C lamp is moved such that the calibrated distance dc is from about 0.75 inches (about 19 mm) to about 1.25 inches (about 31.75 mm). In practice, the cuvette need not be in its irradiation position during calibration, and may, in various embodiments, be temporarily set aside and replaced with one or more light sensors for calibration purposes. In various embodiments, a photosensor capable of measuring UV light intensity is positioned where the cuvette would be located, and the UV-A and UV-C lamps are then moved such that the light intensity is maximized. The blood irradiation device is considered “calibrated” or “tuned” when the UV light intensity, comprising both UV-A and UV-C light, is maximized at the location where the internal flow path of the cuvette will reside. In various embodiments, a separate instrument may be required for measuring intensity of UV-A and UV-C EM radiation if a single sensing instrument is unavailable to measure total light intensity. For example, the ILT-2400 light meter from International Light Technologies, Peabody, Mass., is capable of measuring the intensity of UV-C light from about 230 nm to about 280 nm wavelength. Various UV-A light intensity meters are available from Inspect USA, Inc., Marshall, N.C. In this way, each lamp can be moved independently to maximize the light intensity from each lamp, as evidenced by the UV-A or UV-C specific light intensity meter.

In various embodiments, calibration is electronic, rather than mechanical, by altering the ballast of either or both of the UV-A and UV-C lamps.

### General Methods of Use

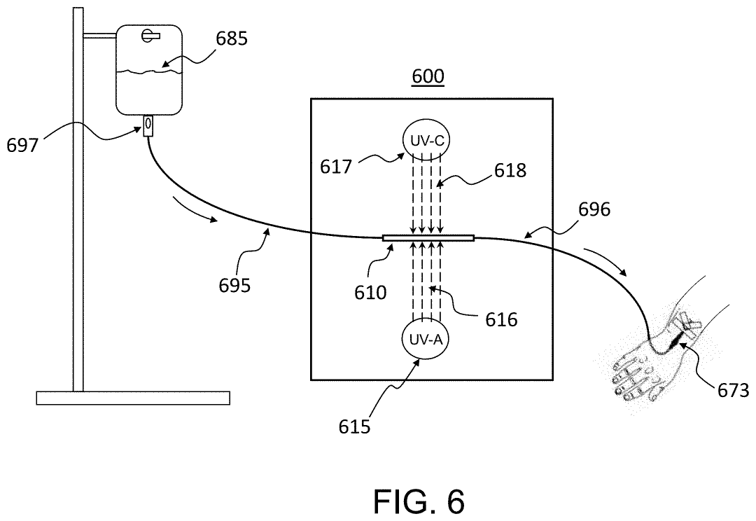

In various embodiments of the present disclosure, a method of using a UBI device is described. In various embodiments, the method comprises moving a fluidic material through a flat quartz crystal flow-through cuvette while simultaneously irradiating the cuvette and the fluidic material as it flows therethrough with simultaneous UV-A and UV-C electromagnetic radiation emanating from opposite sides of the flat cuvette. In various embodiments, the method further comprises calibrating the UV-A and UV-C lamps such that a specific and desired two point source interference pattern is achieved in and around the cuvette. In various embodiments, the UV-A and UV-C lamps are moved relative to the cuvette such that a position is found for each where the total light intensity in the flow path of the cuvette is maximized. For irradiation of a blood sample, the cuvette is connected (e.g., as shown in FIGS. 3 and 6) and the blood sample is irradiated as it passes through the cuvette and to a catheter where the blood is infused into a subject in need thereof. In other embodiments, a blood sample is irradiated as it flows through the cuvette as the blood is transferred from one receptacle (like an IV bag) to another receptacle like a vacuum bottle where it can be stored for future therapeutic use.

### General Embodiments of Medical Treatment Using UBI

In various embodiments of the present disclosure, a UBI treatment method is described. In various embodiments, the UBI treatment method comprises medical treatment of a diseased state in a human or non-human animal subject in need thereof. In various embodiments, a UBI treatment method in accordance with the present disclosure comprises treating at least one of a cancer, an infectious disease, or an autoimmune disease in a human or a non-human subject in need thereof

In various embodiments, a UBI treatment method in accordance with the present disclosure comprises irradiating blood with UV irradiation just prior to the blood being infused or reinfused into the human or non-human animal subject in need thereof. In various embodiments, a blood sample is withdrawn from the subject and then irradiated as it is reinfused back into the same subject. In some ways, this procedure relates to plasmapheresis except that the process involves whole blood rather than blood plasma. The time between the initial withdrawal of the blood sample and the UV irradiation/reinfusion back into the subject may be from minutes to days, weeks, or even months.

In various embodiments, a UBI treatment method in accordance with the present disclosure comprises infusing the human or non-human animal subject in need thereof with compatible blood that was previously irradiated with UV-A and UV-C light as disclosed herein. The blood for this latter method, having been previously irradiated at any time in the past, may have originated from the same subject, or it might be a blood sample obtained from another individual of the same animal species and same blood type (i.e., a compatible blood sample from a blood bank) that has been previously irradiated before being used for therapy. In other words, therapeutic irradiated blood infused into a subject in need thereof may not have originated from the particular subject undergoing treatment. In some ways, this procedure relates to convalescent plasma therapy except that the process involves whole blood, which has been irradiated, rather than blood plasma.

General methods of treatment comprise using the UBI device herein to irradiate a sample of blood simultaneously with UV-A and UV-C electromagnetic radiation as the blood passes through the flat quartz crystal cuvette. In various embodiments, the method further comprises prior calibration of the UV-A and UV-C lamps such that a desired two point source interference pattern is achieved in and around the cuvette, particularly wherein constructive interference points, rather than canceling nodes, are observed within the interior of the cuvette. This desired constructive interference can be confirmed by observing a maximum light intensity within the cuvette or at least a maximum light intensity in the location of the device where the interior of the cuvette will be positioned.

In various embodiments, the flat quartz crystal cuvette is flipped over during the flow-through irradiation process to prevent blood coagulation and film formation on the inside of the cuvette, such as against the inside of one flat side versus the other opposite internal flat side. For example, a blood sample may pass through the cuvette over a period of an hour or several hours, and the cuvette may be flipped over every 5 to 45 minutes. Further, additional cooling fans within the UBI device may be configured to keep the cuvette at less than about 110° F. and preferably less than about 108° F. in order to mitigate blood coagulation in the cuvette. In various embodiments, the blood sample is irradiated as it flows through the cuvette and between containers. Such as from a bag to a vacuum bottle. In various embodiments, the blood sample is irradiated as it flows through the cuvette and into the vasculature of the patient from which the blood sample was withdrawn. In some instances, it is desirable to re-infuse “freshly irradiated” blood, and hence the blood can be irradiated as it is being reinfused.

In various embodiments, a therapeutic treatment of a human or non-human animal subject in need thereof comprises irradiating a compatible blood sample, and then at some later time, infusing that irradiated blood sample into the human or non-human animal subject in need thereof. The original blood sample, prior to UV irradiation, need not have come from the very same human or non-human animal subject undergoing convalescent therapy. Instead, the blood sample may be from a blood bank. In various embodiments, the blood sample from the blood bank is mixed with saline and/or anticoagulants prior to UV irradiation.

In various embodiments, a therapeutic treatment of a human or non-human animal in need thereof comprises irradiating a blood sample previously withdrawn from the human or non-human animal subject in need thereof. For example, a calculated volume of blood is first withdrawn from the subject. The blood sample is optionally mixed with saline and/or anticoagulants. An IV catheter is then placed in the subject from which the sample was taken, (or an initially placed catheter is simply left in place to reduce the number of procedures), and the blood sample, optionally mixed with saline and/or anticoagulant, is reinfused into the subject via the placed catheter, with the cuvette configured in the fluidic line between the withdrawn sample vessel and the placed catheter such that the blood sample is irradiated as it passes through the cuvette and back into the subject. In other words, the procedure looks substantially like an IV infusion, with a bag of fluid to infuse hung up higher than the placed catheter, but where the tubing from the bag of fluid (i.e., the blood sample) and the catheter is spliced and the flow-through cuvette inserted inline so that the blood sample from the bag must flow through the cuvette and irradiated simultaneously by UV-A and UV-C light before it can reenter the vasculature of the subject.

In various embodiments, UBI therapy does not need to include irradiation of all of the blood within a subject in need thereof, but instead just a small sample, such as calculated by the formula herein. Furthermore, irradiation of a blood sample flowing through the cuvette need not eradicate all pathogens within the blood sample, or effect each and every blood cell flowing through the cuvette. Said another way, UBI does not purify the blood nor attempt to treat all the blood, but instead works with the immune and circulatory system of the subject to help these physiological processes function more effectively.

In various embodiments, UBI treatment may provide any one or combination of: increase in erythrocytes, increase in hemoglobin, increase in white blood cells, increase in basophilic granulocytes, increase in lymphocytes, and decrease in thrombocytes. In various embodiments, UBI treatment may effect clotting changes selected from a lowering of fibrin, normalization of fibrinolysis, normalization of fibrin-split products, and lowering of platelet aggregation. In various embodiments, UBI treatment may affect various blood parameters selected from a lowering of full-blood viscosity, a lowering of plasma viscosity, and a reduction of elevated red blood cell aggregation tendencies. In various embodiments, UBI treatment may provide various metabolic changes relating to improvement in oxygen utilization, including, but not limited to, increase in arterial PO₂, increase in venous PO₂, increase in arterial venous O₂ difference (increase O₂ release), increase in peroxide count, fall in oxidation state of blood (increase in reduction state), increase in acid-buffering capacity and a rise in blood pH, reduction in blood pyruvate content, reduction in blood lactate content, improvement in glucose tolerance, and reduction in cholesterol count, transaminases, and creatinine levels. In various embodiments, UBI therapy provides various hemodynamic changes, such as elevation in poststenotic arterial pressure and an increase in volume of circulation. In various embodiments, UBI therapy improves various immune defenses selected from an increase in phagocytosis capability, increase in bactericidal capacity of the blood, and modulation of the immune system overall.

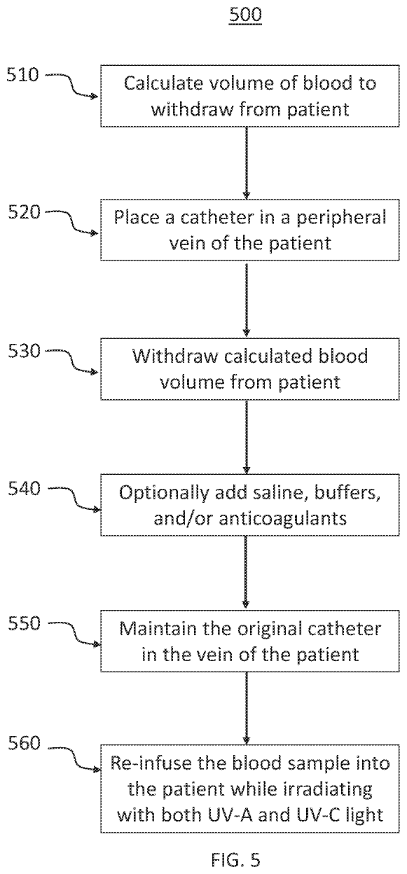

As set forth in the flow chart of FIG. 5, a UBI method 500, such as to treat a disease state in a subject in need thereof comprises:

Step 510, calculating a blood volume to be withdrawn from the subject diagnosed with or being suspected as having a viral infection;

Step 520, placing a catheter in a peripheral vein of the subject;

Step 530, withdrawing the calculated volume of blood from the subject into a container for liquids such as an IV bag;

Step 540, optionally adding saline, buffers, and anticoagulants to the blood sample;

Step 550, maintaining placement of the catheter in the peripheral vein of the subject for the re-infusion process; and

Step 560, re-infusing the blood sample into the subject through a flow-through cuvette irradiated with both UV-A and UV-C light in a UBI device comprising the cuvette, a UV-A lamp configured to irradiate the cuvette and the blood sample flowing therethrough with UV-A light, and a UV-C lamp configured to irradiate the cuvette and the blood sample flowing therethrough with UV-C light.

In various embodiments, the diseased state treated per the method of FIG. 5 is characterized by at least one of a cancer, an infectious disease, or an autoimmune disease.

In various embodiments, the calculated volume of blood to irradiate for a subject in need thereof (in cc's) is determined by multiplying the subject's weight in pounds by 1.5 cc/pound or the subject's weight in kilograms by 3.3 cc/kilogram. However, in various situations, the volume of the blood sample to be withdrawn is capped at 250 cc maximum. Thus, for example, the calculated blood sample to withdraw from a 200 pound man is 200 x 1.5 cc=300 cc. In various examples, this calculated volume is reduced to the 250 cc allowed maximum, and so the blood sample to withdraw from the human male in need of therapy by UBI is 250 cc rather than 300 cc. It should be understood that as new disease states are recognized and treated by the UBI methods herein, and as patient variability is further understood and accounted for, calculated blood withdrawal amounts may vary from this absolute formula of “1.5 cc/pound of body weight with cap of 250 cc.” Further, the blood sample withdrawal calculations might vary for non-human animals, and in particular between species of non-human animals.

In various embodiments, the prior withdrawal of a blood sample from the subject in need thereof is optional. In other words, a previously irradiated blood sample may originate from a blood bank, and may be from some other person or animal entirely. Or, in other embodiments, a sample of blood may be withdrawn from the subject in need thereof and stored for future irradiation with simultaneous UV-A/UV-C and then later infused back into the subject.

In various embodiments, a UBI method to treat a disease state in a subject in need thereof comprises infusing a blood sample in the subject, wherein the blood sample was previously subjected to simultaneous irradiation with UV-A and UV-C light while flowing through a cuvette.

### General Embodiments of a Method of Treating a Viral Infection Caused by a Single-Stranded RNA Virus

In various embodiments of the present disclosure, a method of treating a viral infection in a subject in need thereof is described. The method utilizes the UBI device described herein for the simultaneous irradiation with UV-A and UV-C light, and follows the general flow chart set forth in FIG. 5 for treatment of a disease state. The configuration of the UBI device is as discussed above, with precise calibration of the lamps and adjustment of flow rate through the cuvette during the infusion process. It is important to note that the configuration illustrated in FIG. 6 and discussed below will suffice for the treatment of any cancer, infection, or autoimmune disease, wherein a blood sample is irradiated as it is infused (or reinfused) into the human or non-human animal patient in need thereof

In various embodiments, a viral infection to be treated is caused by a positive-sense ssRNA virus or a negative-sense ssRNA virus. The subject in need of treatment may be symptomatic or asymptomatic. As mentioned, the subject in need thereof may be a human or a non-human animal such as a dog, a cat, or a farm animal.

In various embodiments of the method of viral treatment, the flow-through cuvette comprises a flat quartz crystal cuvette with volume of 1-5 cc and configured with an inlet and an outlet. In various embodiments, the flat quartz crystal cuvette is made from flattening a short length of PQ181 quartz glass tubing and drawing the ends into capillaries configured for connection to flexible tubing. The UV-A lamp is configured to irradiate UV-A light at 330-380 nm. The UV-C lamp is configured to irradiate UV-C light at 245-265 nm. In various embodiments, the amount of blood to withdraw from the subject in need thereof is calculated as 1.5 cc times the subject's bodyweight in pounds, or 3.3 cc times the subject's bodyweight in kilograms, with a maximum blood sample withdrawal capped at 250 cc regardless of the subject's weight. This precise calculation is premised on the subject in need of treatment being a human, and the infection to be caused by a ssRNA virus, such as SARS-CoV-2, recognizing this calculation may need to change for patient variability between patients having the same disease state. In various embodiments, the UV-A lamp and the UV-C lamp are calibrated such that a specific desired two point source interference pattern is obtained. In various embodiments, the UV-A and UV-C lamps are each moved closer to or further from the cuvette until an interference pattern is obtained having constructive interference points located in the cuvette.

With reference now to FIG. 6, the previously withdrawn blood sample 685 is connected with tubing 695 to one end of the cuvette 610, and the opposite end of the cuvette is connected to a placed catheter 673 with another section of tubing 696. The sections of tubing may comprise an IV administration set that has been cut to splice in the cuvette 610. The previously withdrawn blood sample, optionally diluted with saline, buffers, and/or anticoagulants, is infused back into the patient via the placed catheter 673 with the blood sample necessarily passing through the cuvette 610 when it flows from the drip control 697 through the tubing and on to the patient. In various embodiments, the IV fluid drop rate at 697 during the infusion and simultaneous irradiation process is about 60-100 drops per minute. Assuming a drop factor of about 16 for whole irradiated blood, this equates to about 3.75 to about 6.25 mL/min. This rate may be adjusted as needed, such as based on the volume of blood schedules for infusion back into the patient, the dimensions of the cuvette through which the blood must flow, the size of the IV tubing, the length of time desired for the infusion, and the intensity of the UV irradiation.

With continued reference to FIG. 6, the blood sample 685 is irradiated as it flows through the cuvette 610 in the UBI device 600. The cuvette 610 is positioned flat between the UV-A lamp 615, configured to irradiate the cuvette and the blood passing therethrough with UV-A light 616, and the UV-C lamp 617, configured to irradiate the cuvette and the blood passing therethrough with UV-C light 618. The infusion/irradiation process shown in FIG. 6 is continued until the previously withdrawn blood sample is infused back into the patient. As mentioned, if infusing freshly irradiated blood is not important, blood may be irradiated as it flows from one container to another, through the cuvette. Then the irradiated blood in the second container can be reinfused back into the patient.

### Example Medical Treatments

### Infectious Disease Caused by SARS-CoV-2

### Example 1

### Treatment of a 150 Pound Male Testing Positive for SARS-CoV-2:

A. Materials required:

0.9% Sodium chloride injection; a flat quartz crystal flow-through cuvette in accordance with the present disclosure; ≥30cc syringe with 22-18 gauge needle; heparin (at least 20,000 units); alcohol swabs; tape; clamps; IV administration set; scissors; a butterfly catheter; blood collection bottles; and a tourniquet. Typically, the IV administration set is previously cut and the flow-through cuvette spliced in the line. In this way, a cuvette and its associated tubing from the IV administration set connected to the cuvette, can be sterilized, used once, and then disposed.

B. The calculated blood draw is 225 cc, calculated by multiplying 150 pounds×1.5 cc/pound=225 cc.

C. Turn on the UBI device and allow the lamps to warm up. Calibrate the UBI device by turning adjustment screws and axially moving the lamps such that a specific desired two point source interference pattern is achieved.

D. Withdraw the 225 cc blood sample from the infected patient, diluting with saline and heparin as it is transferred to the blood collection bottle.

E. Place a catheter in a peripheral vein of the patient.

F. Connect the spliced IV administration set up between the blood collection bottle and the placed catheter.

G. Place the cuvette into the UBI device 600.

H. Adjust the drip rate and infuse the previously withdrawn blood sample back into the patient while irradiating the blood with both UV-A and UV-C light as the blood flows through the cuvette.

### General Uses of the Blood Irradiation Device

In various embodiments, use of a blood irradiation device in a treatment of a disease state in a human or non-human animal is described. In various embodiments, a blood irradiation device for use in a treatment of a disease state in a human or non-human animal is described. In various embodiments, the disease state is one of a cancer, an infectious disease, or an autoimmune disease.

### General Use:

Use of a blood irradiation device in the treatment of a cancer, an infectious disease, or an autoimmune disease in a human or non-human animal, the blood irradiation device operable to simultaneously irradiate a blood sample with UV-A light and UV-C light incident from a UV-A lamp and a UV-C lamp, respectively, as the blood flows through an interior of a quartz crystal flow-through cuvette, the treatment comprising the simultaneous irradiation of the blood flowing through the interior of the cuvette with both the UV-A and UV-C light prior to infusion into the human or non-human animal in need thereof

Use of a blood irradiation device in the treatment of a cancer, an infectious disease, or an autoimmune disease in a human or non-human animal, the blood irradiation device comprising a flat quartz cuvette having a first flat side and an opposing second flat side, an interior, an inlet, and an outlet configured in fluidic communication for blood flow through the interior; a UV-A lamp configured to irradiate blood flowing through the interior of the cuvette with UV-A light incident upon the first flat side; and a UV-C lamp configured to irradiate the blood flowing through the interior of the cuvette with UV-C light incident upon the opposing second flat side, the treatment comprising simultaneous irradiation of blood flowing through the interior of the cuvette with both the UV-A and UV-C incident light prior to infusion into the human or non-human animal in need thereof

Use of a blood irradiation device in the treatment of a cancer, an infectious disease, or an autoimmune disease in a human or non-human animal, the blood irradiation device comprising a flat quartz cuvette having a first flat side and an opposing second flat side, an interior, an inlet, and an outlet configured in fluidic communication for blood flow through the interior; a UV-A lamp configured to irradiate blood flowing through the interior of the cuvette with UV-A light incident upon the first flat side; and a UV-C lamp configured to irradiate the blood flowing through the interior of the cuvette with UV-C light incident upon the opposing second flat side, the treatment comprising simultaneous irradiation of blood flowing through the interior of the cuvette with both the UV-A and UV-C incident light prior to infusion into the human or non-human animal in need thereof, characterized in that the UV-A light and the UV-C light produce a two point source interference pattern in and around the cuvette.

Use of a blood irradiation device in the treatment of a cancer, an infectious disease, or an autoimmune disease in a human or non-human animal, the blood irradiation device comprising a flat quartz cuvette having a first flat side and an opposing second flat side, an interior, an inlet, and an outlet configured in fluidic communication for blood flow through the interior; a UV-A lamp configured to irradiate blood flowing through the interior of the cuvette with UV-A light incident upon the first flat side; and a UV-C lamp configured to irradiate the blood flowing through the interior of the cuvette with UV-C light incident upon the opposing second flat side, the treatment comprising simultaneous irradiation of blood flowing through the interior of the cuvette with both the UV-A and UV-C incident light prior to infusion into the human or non-human animal in need thereof, characterized in that the UV-A light and the UV-C light produce a two point source interference pattern including constructive interference points in the interior of the cuvette.

### Use for Cancer:

Use of a blood irradiation device in the treatment of a cancer in a human or non-human animal, the blood irradiation device comprising a flat quartz cuvette having a first flat side and an opposing second flat side, an interior, an inlet, and an outlet configured in fluidic communication for blood flow through the interior; a UV-A lamp configured to irradiate blood flowing through the interior of the cuvette with UV-A light incident upon the first flat side; and a UV-C lamp configured to irradiate the blood flowing through the interior of the cuvette with UV-C light incident upon the opposing second flat side, the treatment comprising simultaneous irradiation of blood flowing through the interior of the cuvette with both the UV-A and UV-C incident light prior to infusion into the human or non-human animal in need thereof

Use of a blood irradiation device in the treatment of a cancer in a human or non-human animal, the blood irradiation device comprising a flat quartz cuvette having a first flat side and an opposing second flat side, an interior, an inlet, and an outlet configured in fluidic communication for blood flow through the interior; a UV-A lamp configured to irradiate blood flowing through the interior of the cuvette with UV-A light incident upon the first flat side; and a UV-C lamp configured to irradiate the blood flowing through the interior of the cuvette with UV-C light incident upon the opposing second flat side, the treatment comprising simultaneous irradiation of blood flowing through the interior of the cuvette with both the UV-A and UV-C incident light prior to infusion into the human or non-human animal in need thereof, characterized in that the UV-A light and the UV-C light constructively interfere in the interior of the cuvette.

A blood irradiation device for use in the treatment of a cancer in a human or non-human animal, the blood irradiation device comprising a flat quartz cuvette having a first flat side and an opposing second flat side, an interior, an inlet, and an outlet configured in fluidic communication for blood flow through the interior; a UV-A lamp configured to irradiate blood flowing through the interior of the cuvette with UV-A light incident upon the first flat side; and a UV-C lamp configured to irradiate the blood flowing through the interior of the cuvette with UV-C light incident upon the opposing second flat side, the treatment comprising simultaneous irradiation of blood flowing through the interior of the cuvette with both the UV-A and UV-C incident light prior to infusion into the human or non-human animal in need thereof

A blood irradiation device for use in the treatment of a cancer in a human or non-human animal, the blood irradiation device comprising a flat quartz cuvette having a first flat side and an opposing second flat side, an interior, an inlet, and an outlet configured in fluidic communication for blood flow through the interior; a UV-A lamp configured to irradiate blood flowing through the interior of the cuvette with UV-A light incident upon the first flat side; and a UV-C lamp configured to irradiate the blood flowing through the interior of the cuvette with UV-C light incident upon the opposing second flat side, the treatment comprising simultaneous irradiation of blood flowing through the interior of the cuvette with both the UV-A and UV-C incident light prior to infusion into the human or non-human animal in need thereof, characterized in that the UV-A light and the UV-C light constructively interfere in the interior of the cuvette.

### Use for Infectious Disease:

Use of a blood irradiation device in the treatment of an infectious disease in a human or non-human animal, the blood irradiation device comprising a flat quartz cuvette having a first flat side and an opposing second flat side, an interior, an inlet, and an outlet configured in fluidic communication for blood flow through the interior; a UV-A lamp configured to irradiate blood flowing through the interior of the cuvette with UV-A light incident upon the first flat side; and a UV-C lamp configured to irradiate the blood flowing through the interior of the cuvette with UV-C light incident upon the opposing second flat side, the treatment comprising simultaneous irradiation of blood flowing through the interior of the cuvette with both the UV-A and UV-C incident light prior to infusion into the human or non-human animal in need thereof. The infectious disease is caused by at least one pathogen selected from the group consisting of bacteria, fungi, viruses, mycoplasma, parasites, and combinations thereof

Use of a blood irradiation device in the treatment of an infectious disease in a human or non-human animal, the blood irradiation device comprising a flat quartz cuvette having a first flat side and an opposing second flat side, an interior, an inlet, and an outlet configured in fluidic communication for blood flow through the interior; a UV-A lamp configured to irradiate blood flowing through the interior of the cuvette with UV-A light incident upon the first flat side; and a UV-C lamp configured to irradiate the blood flowing through the interior of the cuvette with UV-C light incident upon the opposing second flat side, the treatment comprising simultaneous irradiation of blood flowing through the interior of the cuvette with both the UV-A and UV-C incident light prior to infusion into the human or non-human animal in need thereof, characterized in that the UV-A light and the UV-C light constructively interfere in the interior of the cuvette. The infectious disease is caused by at least one pathogen selected from the group consisting of bacteria, fungi, viruses, mycoplasma, parasites, and combinations thereof

A blood irradiation device for use in the treatment of an infectious disease in a human or non-human animal, the blood irradiation device comprising a flat quartz cuvette having a first flat side and an opposing second flat side, an interior, an inlet, and an outlet configured in fluidic communication for blood flow through the interior; a UV-A lamp configured to irradiate blood flowing through the interior of the cuvette with UV-A light incident upon the first flat side; and a UV-C lamp configured to irradiate the blood flowing through the interior of the cuvette with UV-C light incident upon the opposing second flat side, the treatment comprising simultaneous irradiation of blood flowing through the interior of the cuvette with both the UV-A and UV-C incident light prior to infusion into the human or non-human animal in need thereof. The infectious disease is caused by at least one pathogen selected from the group consisting of bacteria, fungi, viruses, mycoplasma, parasites, and combinations thereof

A blood irradiation device for use in the treatment of an infectious disease in a human or non-human animal, the blood irradiation device comprising a flat quartz cuvette having a first flat side and an opposing second flat side, an interior, an inlet, and an outlet configured in fluidic communication for blood flow through the interior; a UV-A lamp configured to irradiate blood flowing through the interior of the cuvette with UV-A light incident upon the first flat side; and a UV-C lamp configured to irradiate the blood flowing through the interior of the cuvette with UV-C light incident upon the opposing second flat side, the treatment comprising simultaneous irradiation of blood flowing through the interior of the cuvette with both the UV-A and UV-C incident light prior to infusion into the human or non-human animal in need thereof, characterized in that the UV-A light and the UV-C light constructively interfere in the interior of the cuvette. The infectious disease is caused by at least one pathogen selected from the group consisting of bacteria, fungi, viruses, mycoplasma, parasites, and combinations thereof

### Use for Autoimmune Disease:

Use of a blood irradiation device in the treatment of an autoimmune disease in a human or non-human animal, the blood irradiation device comprising a flat quartz cuvette having a first flat side and an opposing second flat side, an interior, an inlet, and an outlet configured in fluidic communication for blood flow through the interior; a UV-A lamp configured to irradiate blood flowing through the interior of the cuvette with UV-A light incident upon the first flat side; and a UV-C lamp configured to irradiate the blood flowing through the interior of the cuvette with UV-C light incident upon the opposing second flat side, the treatment comprising simultaneous irradiation of blood flowing through the interior of the cuvette with both the UV-A and UV-C incident light prior to infusion into the human or non-human animal in need thereof The autoimmune disease is selected from the group consisting of multiple sclerosis, myasthenia gravis, pernicious anemia, reactive arthritis, rheumatoid arthritis, lupus, chronic Lyme disease, type-1 diabetes, endometriosis, fibromyalgia, sarcoidosis, and combinations thereof

Use of a blood irradiation device in the treatment of an autoimmune disease in a human or non-human animal, the blood irradiation device comprising a flat quartz cuvette having a first flat side and an opposing second flat side, an interior, an inlet, and an outlet configured in fluidic communication for blood flow through the interior; a UV-A lamp configured to irradiate blood flowing through the interior of the cuvette with UV-A light incident upon the first flat side; and a UV-C lamp configured to irradiate the blood flowing through the interior of the cuvette with UV-C light incident upon the opposing second flat side, the treatment comprising simultaneous irradiation of blood flowing through the interior of the cuvette with both the UV-A and UV-C incident light prior to infusion into the human or non-human animal in need thereof, characterized in that the UV-A light and the UV-C light constructively interfere in the interior of the cuvette. The autoimmune disease is selected from the group consisting of multiple sclerosis, myasthenia gravis, pernicious anemia, reactive arthritis, rheumatoid arthritis, lupus, chronic Lyme disease, type-1 diabetes, endometriosis, fibromyalgia, sarcoidosis, and combinations thereof

A blood irradiation device for use in the treatment of an autoimmune disease in a human or non-human animal, the blood irradiation device comprising a flat quartz cuvette having a first flat side and an opposing second flat side, an interior, an inlet, and an outlet configured in fluidic communication for blood flow through the interior; a UV-A lamp configured to irradiate blood flowing through the interior of the cuvette with UV-A light incident upon the first flat side; and a UV-C lamp configured to irradiate the blood flowing through the interior of the cuvette with UV-C light incident upon the opposing second flat side, the treatment comprising simultaneous irradiation of blood flowing through the interior of the cuvette with both the UV-A and UV-C incident light prior to infusion into the human or non-human animal in need thereof The autoimmune disease is selected from the group consisting of multiple sclerosis, myasthenia gravis, pernicious anemia, reactive arthritis, rheumatoid arthritis, lupus, chronic Lyme disease, type-1 diabetes, endometriosis, fibromyalgia, sarcoidosis, and combinations thereof

A blood irradiation device for use in the treatment of an autoimmune disease in a human or non-human animal, the blood irradiation device comprising a flat quartz cuvette having a first flat side and an opposing second flat side, an interior, an inlet, and an outlet configured in fluidic communication for blood flow through the interior; a UV-A lamp configured to irradiate blood flowing through the interior of the cuvette with UV-A light incident upon the first flat side; and a UV-C lamp configured to irradiate the blood flowing through the interior of the cuvette with UV-C light incident upon the opposing second flat side, the treatment comprising simultaneous irradiation of blood flowing through the interior of the cuvette with both the UV-A and UV-C incident light prior to infusion into the human or non-human animal in need thereof, characterized in that the UV-A light and the UV-C light constructively interfere in the interior of the cuvette. The autoimmune disease is selected from the group consisting of multiple sclerosis, myasthenia gravis, pernicious anemia, reactive arthritis, rheumatoid arthritis, lupus, chronic Lyme disease, type-1 diabetes, endometriosis, fibromyalgia, sarcoidosis, and combinations thereof

In the detailed description, references to “various embodiments”, “one embodiment”, “an embodiment”, “an example embodiment”, etc., indicate that the embodiment described may include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, it is submitted that it is within the knowledge of one skilled in the art to affect such feature, structure, or characteristic in connection with other embodiments whether or not explicitly described. After reading the description, it will be apparent to one skilled in the relevant art(s) how to implement the disclosure in alternative embodiments.

Steps recited in any of the method or process descriptions may be executed in any order and are not necessarily limited to the order presented. Furthermore, any reference to singular includes plural embodiments, and any reference to more than one component or step may include a singular embodiment or step. Also, any reference to attached, fixed, connected, coupled or the like may include permanent (e.g., integral), removable, temporary, partial, full, and/or any other possible attachment option. Any of the components may be coupled to each other via friction, snap, sleeves, brackets, clips, or other means now known in the art or hereinafter developed. Additionally, any reference to without contact (or similar phrases) may also include reduced contact or minimal contact.

Benefits, other advantages, and solutions to problems have been described herein with regard to specific embodiments. However, the benefits, advantages, solutions to problems, and any elements that may cause any benefit, advantage, or solution to occur or become more pronounced are not to be construed as critical, required, or essential features or elements of the disclosure. The scope of the disclosure is accordingly to be limited by nothing other than the appended claims, in which reference to an element in the singular is not intended to mean “one and only one” unless explicitly so stated, but rather “one or more.” Moreover, where a phrase similar to ‘at least one of A, B, and C’ or ‘at least one of A, B, or C’ is used in the claims or specification, it is intended that the phrase be interpreted to mean that A alone may be present in an embodiment, B alone may be present in an embodiment, C alone may be present in an embodiment, or that any combination of the elements A, B and C may be present in a single embodiment; for example, A and B, A and C, B and C, or A and B and C.

All structural, chemical, and functional equivalents to the elements of the above-described various embodiments that are known to those of ordinary skill in the art are expressly incorporated herein by reference and are intended to be encompassed by the present claims. Moreover, it is not necessary for an apparatus or component of an apparatus, or method in using an apparatus to address each and every problem sought to be solved by the present disclosure, for it to be encompassed by the present claims. Furthermore, no element, component, or method step in the present disclosure is intended to be dedicated to the public regardless of whether the element, component, or method step is explicitly recited in the claims. No claim element is intended to invoke 35 U.S.C. 112(f) unless the element is expressly recited using the phrase “means for.” As used herein, the terms “comprises”, “comprising”, or any other variation thereof, are intended to cover a non-exclusive inclusion, such that a chemical, chemical composition, process, method, article, or apparatus that comprises a list of elements does not include only those elements but may include other elements not expressly listed or inherent to such chemical, chemical composition, process, method, article, or apparatus.F2

1. An ultraviolet blood irradiation (UBI) device comprising:
a cuvette having an interior, an inlet, and an outlet configured in fluidic communication for blood flow through the interior;
a UV-A lamp and a UV-C lamp configured to simultaneously irradiate blood flowing through the interior of the cuvette with both incident UV-A light and UV-C light.

2. The device of claim 1, wherein the cuvette comprises a first flat side and a second flat side opposite the first flat side, and wherein the cuvette is positioned between the UV-A lamp and the UV-C lamp such that the UV-A lamp irradiates the first flat side of the cuvette with UV-A light and the UV-C lamp irradiates the second flat side of the cuvette opposite the first flat side of the cuvette with UV-C light.

3. The device of claim 2, wherein the UV-A light and the UV-C light produce a two point source interference pattern in and around the cuvette.

4. The device of claim 3, wherein the two point source interference pattern provides constructive interference points in the interior of the cuvette.

5. The device of claim 4, wherein the UV-A lamp is separated from the first flat side of the cuvette by a distance da, and wherein the UV-C lamp is separated from the second flat side of the cuvette by a distance dc.

6. The device of claim 5, wherein the distance da is from about 0.75 inches (about 19 mm) to about 1.25 inches (about 31.75 mm), and wherein the distance dc is from about 0.75 inches (about 19 mm) to about 1.25 inches (about 31.75 mm).

7. The device of claim 1, wherein the cuvette comprises a flat quartz crystal cuvette, and wherein the inlet and the outlet comprise drawn capillary ends dimensionally configured to connect to flexible tubing.

8. The device of claim 1, wherein the UV-A lamp irradiates electromagnetic radiation having a wavelength from about 330 nm to about 380 nm, and wherein and the UV-C lamp irradiates electromagnetic radiation having a wavelength from about 245 nm to about 265 nm.

9. The device of claim 8, wherein the UV-C light irradiates monochromatic electromagnetic radiation at about 254 nm.

10. The device of claim 1, wherein each of the UV-A and UV-C lamps are mounted in separate lamp frames that are each movable relative to the cuvette by adjustment screws.

11. The device of claim 1, further comprising first and second housings movable relative to one another through a hinge, the first housing enclosing the UV-A lamp, the second housing enclosing the UV-C lamp, wherein the cuvette is disposed between the housings.

12. A method of treating a cancer, an infectious disease, or an autoimmune disease in a human or non-human animal subject in need thereof, the method comprising: simultaneously irradiating a blood sample with UV-A light and UV-C light incident from a UV-A lamp and a UV-C lamp, respectively, as the blood flows through an interior of a quartz crystal flow-through cuvette; and infusing the blood sample thus irradiated into the subject.

13. The method of claim 12, further comprising withdrawing the blood sample from the subject prior to simultaneously irradiating the blood sample with UV-A and UV-C light.

14. The method of claim 12, further comprising adding saline, buffers, or an anticoagulant to the blood sample prior to simultaneously irradiating the blood sample with UV-A and UV-C light.

15. The method of claim 12, wherein the cuvette is flat, having a first flat side and a second flat side opposite the first flat side, and wherein the cuvette is positioned between the UV-A lamp and the UV-C lamp such that simultaneously irradiating the blood sample with UV-A and UV-C light further comprises irradiation of the first flat side with UV-A light and irradiation of the second flat side opposite the first flat side with UV-C light while the blood sample passes through the interior of the cuvette, characterized in that the UV-A light and the UV-C light produce a two point source interference pattern in and around the cuvette.

16. The method of claim 15, wherein the UV-A light has wavelength from about 330 nm to about 380 nm and the UV-C light has wavelength from about 245 nm to about 265 nm.

17. The method of claim 15, further comprising moving at least one of the UV-A lamp and UV-C lamp relative to the cuvette prior to simultaneously irradiating the blood sample, such that the two point source interference pattern includes constructive interference points in the interior of the cuvette.

18. The method of claim 17, wherein moving at least one of the UV-A lamp and UV-C lamp relative to the cuvette further comprises obtaining a maximum light intensity within the interior of the cuvette.

19. The method of claim 12, wherein the blood flows through the interior of the quartz crystal cuvette at a flow rate from about 3.75 mL/min to about 6.25 mL/min.

20. The method of claim 12, wherein the infectious disease is caused by SARS-CoV-2.
